(19) United States
(12) Patent Application Publication
(10) Pub. No.: US 2015/0047635 A1
(43) Pub. Date: Feb. 19, 2015

(54) DUCKBILL VALVE AND INHALATION DEVICE INCLUDING SUCH A VALVE

(71) Applicant: PROTECSOM, Valognes (FR)

(72) Inventor: Thierry Poree, Valognes (FR)

(21) Appl. No.: 14/386,642

(22) PCT Filed: Mar. 15, 2013
(86) PCT No.: PCT/EP2013/055321

§ 371 Date: Sep. 19, 2014

(30) Foreign Application Priority Data
Mar. 22, 2012 (FR) 1252555

(51) Int. Cl. A61M 15/00; F16K 15/14

(52) U.S. Cl. CPC A61M 15/0016; A61M 15/0021; A61M 15/0065; F16K 15/147
USPC 128/203.12; 137/846

(57) ABSTRACT

The invention relates to a valve (300) comprising a base (301) in the form of a ring defining a central opening (309), as well as an upper surface (302) and a lower surface (303), both of which are planar and converge toward each other from the base (301) in the direction opposite said base, and characterized in that the upper and lower surfaces (302, 303) together form an angle (Ang) of at least 60°, and in that the free edges (304, 305) thereof define a slit (306) which is open when the valve is at rest.

The present invention is related to a valve for an inhalation device for drug substances, and an inhalation device comprising such a valve.

Active therapeutic substances can be administered by inhalation in a patient's lungs. Those substances can be administered by means of a metered dose inhaler (MDI), in which a propelling gas generates a cloud of the active substance. Several drugs against asthma, or for curing pathologies such as bronchopulmonary diseases, asthma, bronchopathies, or bronchiolitis, are administered using MDIs.

The use of an inhalation chamber, or spacer, has long been recognized as easing and improving the intake of medication, in particular increasing the deposit of substances in bronchi and reducing the flow speed. Indeed, when such an inhalation device is absent, coordination between the triggering of the MDI and inspiration is crucial. However, this coordination is hard to perform for the patient, more particularly for children. MDIs are shaped as a chamber defining an internal volume in which aerosol is propelled through an opening provided at an end of the chamber and on which an MDI is fit. At another end of the chamber, there is provided an opening in communication with the mouth of the patient via a linker, generally a tubular portion, such as a mouthpiece that patients can directly take in they mouth. It is also possible to connect a mask on this linker, especially in the case of devices designed for young children. The active substance is propelled in the inhalation chamber by pressing on the MDI. Since the patient inspires through the linker, the active substance is transported to the patient's bronchi by a flow exiting the chamber which is generated by an inspiratory flow. During expiration, the flow must not go back inside the chamber. In practice, a unidirectional valve, called inspiratory valve, is provided between the inhalation chamber and the patient's mouth, at the opening of the chamber which opens toward the linker, to manage the flow of active substances, more particularly, to allow the flow to exit the chamber at an adequate speed and to prevent the flow to enter the chamber during expiration. An opening provided in the linker, upstream from the valve with respect to the patient, allows the exit of the expiratory flow.

Regarding unidirectional valves, several devices comprise an inhalation chamber using so-called duckbill valves. This type of valve, well known to the skilled person, comprise a ring-shaped base defining a central opening of a given diameter. The valve is to be crossed by a flow which penetrates therein through that central opening. It further comprises two oblique walls defining respectively an upper surface and a lower surface, both of which are planar and converge toward each other from the base in the direction opposite said base until their free edges join together and touch themselves in order to form a sharp roof. The upper and lower surfaces together form an acute angle. At the junction of both free edges, there is provided a slit which is transversal to the central axis crossing the ring-shaped base. The sides of the valve, on each side of the upper and lower surfaces, are convexly curved. At rest, the free edges of the upper and lower surfaces are joined together. The valve's slit is thus closed. When used in an inhalation device, the valve's base is secured in a housing provided in the linker between the chamber and the patient.

During the patient's inspiratory phase, under the negative pressure due to inspiration, the free edges of the upper and lower surfaces move away one from the other, thereby opening the slit to allow the passage of the flow exiting the inhalation chamber. At the end of the inspiratory phase, the free edges are again joined together and the slit shuts itself. During the following expiratory phase, the slit is maintained closed due to expiratory pressure, thereby preventing any flow to pass across the valve, especially from the inside of the inhalation chamber. Such a valve is described, for example, in patent application US 200710235028.

This type of valve is made of a soft material, such as silicone. The thickness of the walls and planar surfaces needs to be small in order to provide low resistance during inspiration. Unfortunately, those valves have a significant inconvenient since they are prone to overturn easily when the patient outbreathes too strongly or in case of cough. In this case, the walls of the valve go through the ring-shaped base toward the inside of the chamber, thereby resulting in a significant dysfunction in which the opening closes during the next inspiratory phase and opens during the expiratory phase. If, on the other hand, the thickness of the walls is increased, resistance during inspiration increases too.

The object of the present invention is to provide a duckbill valve, especially for inhalation devices for drug substances, which offers at the same time weak resistance during inspiration and strong resistance during expiration and which does not cause dysfunction. In particular, the valve according to the invention must not overturn during expiration.

Therefore, the invention is directed to a duckbill valve comprising a base in a ring shape defining a central opening, an upper surface and a lower surface, both of which are planar and converge toward each other from the base in the direction opposite said base, and characterized in that the upper and lower surfaces together form an angle of at least 60° and in that the free edges thereof define a slit which is open when the valve is at rest. According to the invention, the terms “valve at rest” are intended to mean a valve on which there is not exerted any pressure, such as the pressure during inspiration and expiration when the valve is used in an inhalation device.

The valve, according to the invention, offers weak resistance to opening when pressure is applied on the internal faces of the upper and lower surfaces because it is constantly open at rest. On the contrary, the slit shuts itself totally when pressure is exerted on the external faces of the upper and lower surfaces. When the slit is closing, the free edges of both surfaces touch themselves along their whole length. This closure is enabled by the angle formed between both surfaces.

Advantageously, the free edges of the upper and lower surfaces are cut in a curved and concave fashion. Having the free edges cut in a curved, or curvilinear, and concave fashion eases the closing of the slit, i.e. they can get closer until they join themselves together, when pressure is exerted on the external faces of the upper and lower surfaces.

According to an embodiment, the cross-section of the free edges is beveled.

According to an embodiment, the central opening comprises a circular or oval cross-section.

According to an embodiment, the valve is made of an elastomeric material, preferably of silicone.

The invention is further directed to an inhalation device for drug substances, of the type comprising an inhalation chamber and a linker between the chamber and a patient, such as a mouthpiece, characterized in that it comprises a valve according to the invention as defined above, in particular a duckbill valve comprising a base in a ring shape defining a central opening, an upper surface and a lower surface, both of which are planar and converge toward each other from the base in the direction opposite said base, and characterized in that the upper and lower surfaces together form an angle of at least 60° and in that the free edges thereof define a slit which is open when the valve is at rest.

According to an embodiment, the linker between the chamber and the patient comprises an opening having a window comprising apertures which open on a tubular portion in communication with the outside of said device and a valve capable of moving between two positions, a first position in which it abuts against the window, thereby blocking the flow across the window, and a second position in which it is away from the window.

According to an embodiment, the valve is flat and comprises a rod which is inserted in a housing provided along the central axis of the window, said rod being free to slide within said housing.

Features of the invention described above, and further features of the present disclosure, will become apparent from the following detailed description, taken in combination with the appended drawings, in which:

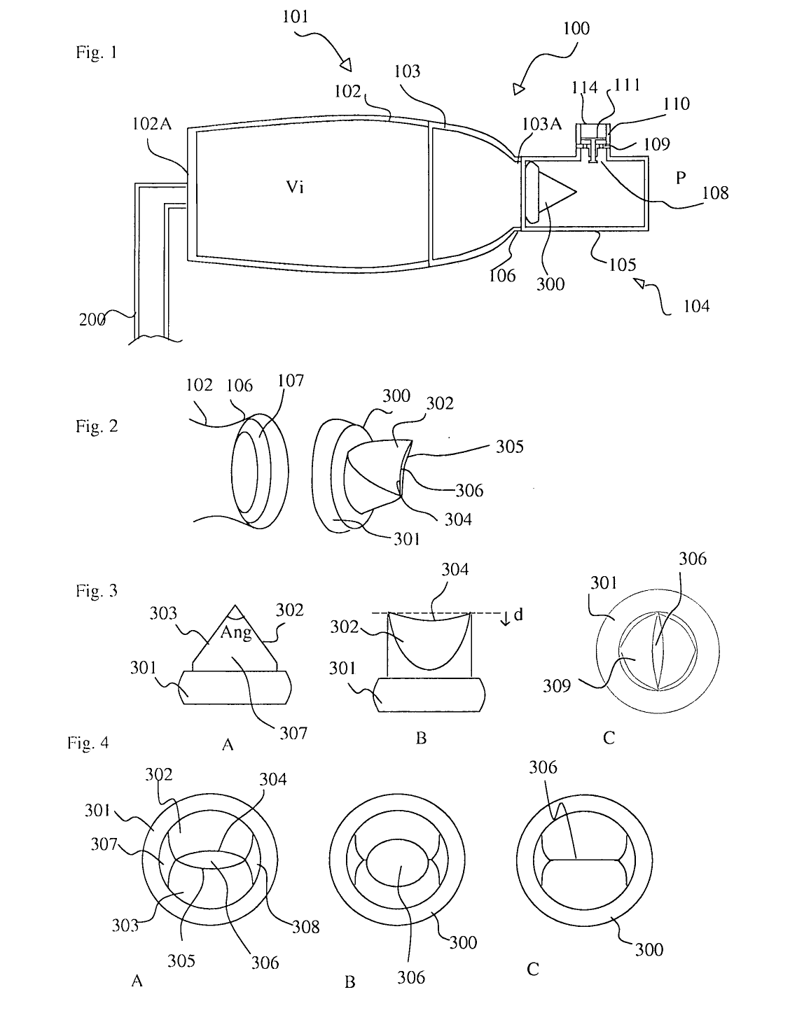

FIG. 1 is a side view illustrating an inhalation device, according to an embodiment;

FIG. 2 is a perspective view illustrating a valve and a neck of an inhalation device, according to an embodiment;

FIGS. 3 A and B are side views illustrating respectively a valve, according to an embodiment;

FIG. 3 C is a bottom view illustrating a valve, according to an embodiment;

FIGS. 4 A, B, C, are bottom views illustrating a valve, according to an embodiment, at rest (FIG. 4A), during an inspiration phase in use in an inhalation device (FIG. 4 B) during an expiration phase in use in an inhalation device (FIG. 4 C);

FIG. 5 is an exploded view illustrating an expiration valve, according to an embodiment;

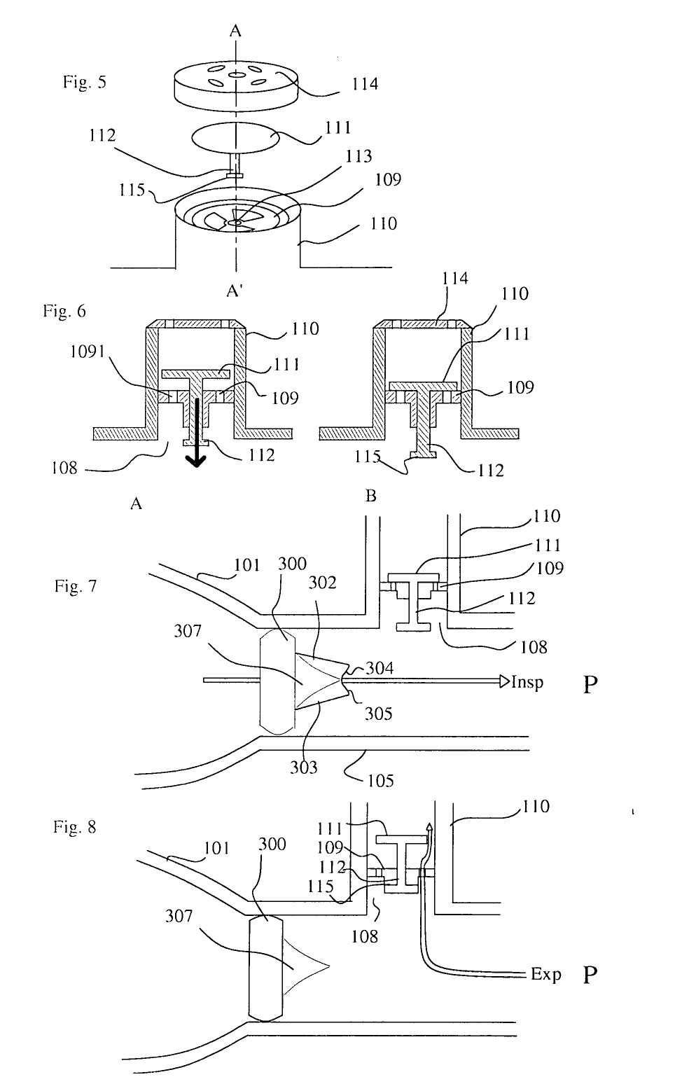

FIGS. 6 A and B are cross-section views along A-A′ axis illustrating an expiration valve in use, according to an embodiment; and

FIGS. 7 and 8 illustrate the workings of a duckbill valve and of an expiration valve, according to an embodiment, during an inspiratory phase (FIG. 7) and during an expiratory phase (FIG. 8).

In FIG. 1, there is shown an inhalation device 100, especially for administering inhaled medication for treating bronchopulmonary diseases, comprising an inhalation chamber 101 composed, according to this embodiment, of two substantially tubular parts 102, 103, one of them fitting in the other one. The inhalation chamber can of course be provided in another form, such as two frustoconical parts or one tubular part. A first end of the chamber 102 A is connected to a MDI 200 containing an active substance and a second end 103 A opposing the first end is connected to a linker 104 between chamber 101 and patient P, precisely a mouthpiece 105. The linker 105 may be a facial mask. The linker 105 fits on a neck 106 provided at the end 103A of the chamber. It is easily dismountable.

A duckbill valve 300 is provided upstream of patient P with respect to the internal volume Vi of chamber 101. This valve 300 is an unidirectional and inspiratory valve enabling a flow transporting medicine particles from inside the chamber 101 toward the patient during the inspiratory phase of patient P.

A housing 107 for the duckbill valve 300 is provided in the neck 106 of the device 100. It may consist, for example, of a groove or of a flat part (FIG. 2).

The duckbill valve 300 according to the invention is more thoroughly illustrated in FIGS. 2, 3 and 4. It comprises a base 301, in the shape of a ring or of a collar, defining a central opening 309 of a given diameter (FIG. 3 C). The base 301 is designed for seating in the housing 107 of the device 100 (FIG. 2).

The valve 300 comprises two oblique walls forming an upper surface 302 and a lower surface 303, which are both planar. Sides 307, 308 on both sides of the upper and lower surfaces 302, 303 are convexly curved.

The upper and lower surfaces 302, 303 converge toward each other from the base in the direction opposite said base, and form an angle Ang of at least 60°. When viewed from aside, facing a side 307 or 308, the valve 300 has a duckbill shape or a roof shape (FIG. 3A).

The surfaces 302, 303 have a semi-elliptical shape.

The free edges 304, 305 of the upper and lower surfaces 302, 303 are curved and concave. The concavity is directed toward the base 301 (FIG. 3 B, arrow d) and is weak, from about 0.2 to 1 mm.

Since they are cut in a curved and concave manner, the free edges 304, 305 do not touch themselves along their whole length and define a slit 306 which is transversal to the central axis going across the ring-shaped base 301. The slit 306 is visibly open at rest, i.e. when not in use, as shown in FIGS. 3C and 4A.

The valve 300 is made by molding of an elastomeric material, preferably of silicone. Cutting the edges in a rounded fashion is performed after unmolding.

As can be seen in FIG. 1, the linker 105 between the chamber 101 and patient P further comprises an opening 108 for the outflow of the patient's expiratory flow when he or she expires in the device 100. In the opening 108, there is provided a window 109 which comprises one or more apertures and opens in a tubular portion 110 which is itself in communication with the outside of the device 100. A detailed view of the tubular portion 110 is shown in FIG. 5.

A flat valve 111, which is circular and soft, is provided in the tubular portion 110 for sealing the apertures of window 109 during the inspiratory phase. The flat valve 111 comprises a central rod 112 which is inserted in a housing 113 provided in the central axis of the window 109 (FIG. 5). The tubular portion 110 is covered by a hat 114 comprising apertures. The hat 114 is simply fit on the portion 110 and is thus easily dismountable.

The rod 112 is free to slide along the axis of the housing 113. The valve 111 can thus move between two positions, a first position, called dosed position, in which its flat portion abuts against the window 109 giving access to the mouthpiece 105 (FIG. 6A), thereby blocking the passage of the flow across the apertures 1091 of the window 109 and another position, called open position, in which it is away from window 109 in order to let free the passage of the flow across the window (FIG. 6 B). The rod 112 comprises a foot 115 abutting against the housing 113. The foot 115 helps avoid that the rod leaves the housing 113 when it is in the open position.

FIGS. 7 and 8 illustrate the workings of the valve 300 as well as the expiratory valve 111, within the inhalation device 100.

When the patient P inspires, there is created a low pressure in the linker 105. The pressure is strong on the internal faces of the upper and lower surfaces 302, 303. The free edges 304, 305 get away from each other and the slit 306 of the valve 300 opens itself more than at rest. The valve 300 offers weak resistance to inspiration because of its constant opening when at rest. The inspiratory flow, represented by an arrow, which transports the drug substance, can cross the valve 300 from chamber 101 toward patient P (FIG. 7).

When the valve 300 opens to allow inspiratory flow, the valve 111 places itself in a position in which it closes the passage across window 109 by abutting against it (FIG. 7).

Conversely, during the next phase in which the patient expires, in the linker 105, pressure is exerted on the external faces of the upper and lower surfaces 302, 303. The slit 306 is closed completely by approaching and joining of the free edges 304, 305. The free edges 304, 305 touch themselves along their whole length (FIG. 8). This closure is favored by having the angle Ang formed between the lower and upper surfaces 302, 303 greater than or equal to 60°. The curved and concave shape of the free edges 304, 305 also eases the closing of the slit 306.

When the valve 300 closes, the rod 112 of the valve 111 slides along axis 113 and the valve 11 moves away from the window 109 and places itself in a position in which the expiratory flow is enabled (see the arrow) across the window 109 toward the outside. The foot 115 stops the movement of rod 112 when it abuts against the walls of the housing 113 (FIG. 8).

The valve 300 and the valve 111 are dismountable and washable.

1. A duckbill valve comprising a ring shaped base defining a central opening, an upper surface and a lower surface, both of which are planar and converge toward each other from the base in the direction opposite said base, wherein the upper and lower surfaces together form an angle of at least 60° and wherein free edges thereof define a slit which is open when the valve is at rest.

2. The valve according to claim 1, wherein the free edges of the upper and lower surfaces are cut in a curved and concave fashion.

3. The valve according to claim 1, wherein a cross-section of the free edges is beveled.

4. The valve according to claim 1, wherein the central opening comprises a circular or oval section.

5. The valve according to claim 1, wherein the valve is made of an elastomeric material.

6. An inhalation device for delivery of drugs to a patient, comprising an inhalation chamber and a linker between the inhalation chamber and the patient, comprising a valve according to claim 1.

7. The inhalation device according to claim 6, wherein the linker between the inhalation chamber and the patient comprises an opening on which is mounted a window comprising apertures which open on a tubular portion in communication with the outside of said device and a valve capable of moving between two positions, a first position in which it abuts against the window, thereby blocking the flow across the window, and a second position in which it is away from the window.

8. The inhalation device according to claim 7, wherein the valve is flat and comprises a rod which is inserted in a housing provided along the central axis of the window, said rod being free to slide within said housing.

9. The valve of claim 5, wherein the elastomeric material is silicone.

10. The inhalation device of claim 6, wherein the linker is a mouthpiece.
